(19) United States
(12) Patent Application Publication
(10) Pub. No.: US 2017/0065971 A1
(43) Pub. Date: Mar. 9, 2017

(54) IMPROVED SAMPLE TUBE WITH TRANSPARENT TIP HAVING PARTICULAR UTILITY FOR NUCLEIC ACID AMPLIFICATION

(71) Applicant: Streck, Inc., LaVista, NE (US)

(72) Inventors: Matthew R. Kreifels, Omaha, NE (US); Scott E. Whitney, Lincoln, NE (US); James Dowling, New Boston, NH (US); Troy Just, Lincoln, NE (US)

(21) Appl. No.: 15/123,396

(22) PCT Filed: Jun. 30, 2014
(86) PCT No.: PCT/US2014/044867

§ 371 Date: Sep. 2, 2016

Related U.S. Application Data
(60) Provisional application No. 61/947,697, filed on Mar. 4, 2014.

(51) Int. Cl. B01L 3/00; C12Q 1/68

(52) U.S. Cl. CPC B01L 3/5082; C12Q 1/686; B01L 2300/0848; B01L 2300/123; B01L 2300/0832; B01L 2300/168

(57) ABSTRACT

An improved sample tube that includes a body portion having a longitudinal axis and a wall generally circumscribing the longitudinal axis. The body portion terminates in a distal tip having a dimple. The body portion includes at least one transparent portion (e.g., at the distal tip) that is adapted for transmitting light. The wail is configured for elastic deformation along at feast a portion of its length, including in a direction that is generally transverse to the longitudinal axis, so that it compressively and resiliently deforms and engages a wail defining an opening in a sample block of a PGR amplification instrument. The tube may be made by injection molding a polymeric material including a thermoplastic.

### FIELD OF THE INVENTION

The present invention relates generally to container's, and more particularly to unique resilient polymeric sample tubes with a transparent tip for nucleic acid amplification and real-time optical analysis.

### BACKGROUND OF THE INVENTION

There is a need for sample holders that are thermally efficient in the manner in which heat is delivered to a contained sample, removed from a contained sample, or both. This is particularly acute in the field of polymerase chain reaction (PCR) amplification of nucleic acid (e.g., DNA amplification). In such applications, samples are exposed to a dynamic heating and cooling protocol. Successful amplification often relies upon time dependent heat transfer. As a result, the efficiency of such operations can be limited when the mass, volume, or length of heat transfer of a sample is such that it impedes heat transfer within it, and to and from it.

One approach to sample tubes for amplification of nucleic acid has been to employ glass capillaries. While useful, the risk of breakage during use and the inability to deform such glass tubes during an amplification process make the use of glass capillaries an undesirable option. Another approach has been to employ polymeric sample vessels. However, the polymeric material may not provide sufficient heat transfer to substances within the tubes and may also fail to provide sufficient elasticity to be compressed as necessary during the amplification process. Further, the clarity of polymeric tubes has been insufficient for efficient light transfer in certain types of PCR protocols. In addition, molding processes for formation of the polymeric tubes have traditionally been unable to produce tubes having wall thicknesses that are sufficiently thin for effective heat transfer. Attempts to form such thin walls by injection molding frequently result in weak spots and openings along the tube body. Examples of such polymeric and glass sample holders include those in U.S. Pat. Nos. 5,225,165; 5,571,479; 5,604,101; 5,721,136; 5,863,791; 5,958,349; 6,015,534; 6,159,727; 6,312,886; 6,783025; 7,255,833; and 7,749,452. One particular example of an improved tube is disclosed in published United States Application No. 20120269703; see also, U.S. Pat. Nos. D690,025; D659,848 both incorporated by reference herein for all purposes.

The increased interest in real-time PCR analysis has presented additional challenges in developing suitable polymeric tubes. One difficulty that has been encountered has been to balance the competing needs for the ability to achieve rapid heat exchange between a PCR amplification instrument and an analyte, and the ability to optically gather data about the analyte. One approach to achieving rapid amplification of a nucleic acid is disclosed in co-pending published U.S. patent application Ser. No. 12/918,914, incorporated by reference for all purposes. Because of the need for rapid heat exchange along the length of a sample tube, it may be impractical for some applications to locate optical sensing hardware transverse of the sample tube within an instrument. One approach to obviate this is to employ optical sensing hardware beneath a sample tube within an instrument. This is the subject of co-pending U.S. application Ser. Nos. 13/833,349 (filed Mar. 15, 2013) and 61/840,755 (filed Jun. 28, 2013), both incorporated by reference for all purposes. Unfortunately, due to relatively small sample volumes and associated relatively small amounts of detectable light (e.g., from a luminescing agent, a fluorophore or other light emitting agent), the ability to detect an analyte of interest can be compromised depending (for example) upon the choice of sample tube material, the sample tube geometry, and/or the technique used for the manufacture of the tube.

There is thus a need for an improved polymeric sample tube that provides for both sufficient heat transfer and sufficient elasticity for use in amplification processes that require compression of the tube during use (e.g., such as is taught in co-pending U.S. patent application Ser. No. 12/918,914). There is also a need for such an improved polymeric sample tube to provide at least some amount of optical transparency for real-time PCR analysis of a sample, such as an analysis that may be used in order to amplify and quantify a targeted nucleic acid (e.g., DNA or RNA) molecule. Moreover, there are competing technical demands that often result from efforts to provide a tube that is both sufficiently optically transparent for real-time PCR (especially for small volume samples from which the amount of detectable fluorophore tends to be relatively small), and also provides the necessary heat exchange characteristics for effective sample amplification. It would be attractive to have a tube that meets both the optical transparency and the heat exchange needs of real-time PCR applications, especially in instances when sample volumes are relatively small and/or the area over which optical detection is conducted is relatively small.

### SUMMARY OF THE INVENTION

The present teachings meet one or more of the above needs by providing a sample tube comprising a body portion having a longitudinal axis and an outer wall generally circumscribing the longitudinal axis, the body portion including a tapered sample portion having a first outer wall dimension and including a closed substantially transparent distal tip, the sample portion being generally elongated along the longitudinal axis and being configured for elastic deformation along at least a portion of its length. The substantially transparent distal tip is preferably configured to include a concave dimple that projects generally inwardly within the interior of the sample portion and has a dimple height relative to a tip end.

The teachings herein further provide for an improved sample tube, and particularly a polymeric sample tube that includes a closure portion, a strap integrally connected to the closure portion and being configured for defining a living hinge, and a body portion having a longitudinal axis and an outer wall generally circumscribing the longitudinal axis. The body portion is integrally and hingedly connected with the closure portion by way of the strap. The body portion includes a head portion that has an opening through which a sample is dispensed, and a tapered sample portion having a first outer wall dimension. The body portion also includes at least one transparent portion that is adapted for transmitting light for excitation of a luminescing agent, a fluorophore or some other light emitting agent, and is also adapted for transmitting light emitted by a luminescing agent, a fluorophore or some other light emitting agent that has been excited and is coupled with an analyte of interest. For instance, the body portion may have a closed substantially transparent distal tip that is located at an end of the sample tube that is remote from the head portion. A wall structure may include an outer wall and an inner wall structure for defining a hollow cavity within which the sample resides as a sample volume after it is dispensed through the head portion. The sample portion is generally elongated along the longitudinal axis and is configured for elastic deformation along at least a portion of its length, including in a direction that is generally transverse to the longitudinal axis. In this manner, it is envisioned that at least a portion of the wall structure compressively and resiliently deforms and engages a wall defining an opening in a sample block of a PCR amplification instrument, and a first outer wall dimension of the sample portion reduces to a smaller second outer wall dimension.

The substantially transparent distal tip may be configured to include at least one concave dimple that projects generally inwardly within the interior of the sample portion and has a dimple height relative to a tip end. It will be seen that the portion of the tube wall that defines the dimple will have a generally constant wall thickness. Thus, there will be both a projection of the tube wall into the sample portion, and a depression in the exterior of the tube tip.

The tube may be a molded structure (e.g., a structure made by injection molding) fabricated from a polymeric material including a thermoplastic that exhibits a melt flow rate of about 35 to about 60 g/10 min (per ASTM D-1238-10), a flexural modulus of about 900 to about 1400 MPa (per ASTM D-790A-10 (reported as 2% secant)), and a haze (per ASTM D-1003-11e1: for a section of about 1.1 mm thickness) below about 12%. The tube may be a molded structure fabricated from a material including a polyolefin that exhibits a melt flow rate of about 40 to about 55 9/10 min (per ASTM 0-1238-10), a flexural modulus of about 1000 to about 1200 MPa (per ASTM D-790-10 (reported as 2% secant)), and a haze (per ASTM 0-1003-11e1; for a section of about 1.1 mm thickness) below about 9%. By way of example, the tube may be a molded structure fabricated from a polymer consisting essentially of (e.g., it includes at least about 90 percent by weight of) a random polypropylene copolymer. The transparent portion of the tube will exhibit a haze (per ASTM 0-1003-11e1) below about 12%, 9% or even below about 6%.

The teachings herein also envision use of the sample tube in an instrument for performing steps of PCR amplification of an analyte (e.g., a nucleic acid such as DNA or RNA), and real-time analysis of the analyte based upon light emitted from one or more excited light emitting agents contained within the sample tube and being associated with an amplified analyte of interest. For example, one approach is to perform the real-time analysis using steps of transmitting light through the tip of the sample tube; that is, light for exciting a light emitting agent and/or light emitted by a light emitting agent is transmitted through the tube tip and based solely upon the light transmitted through the tube tip.

As will be seen, such a tube in accordance with the present teachings offers a unique approach to handling a material, and especially a biological sample. It is seen that, particularly as employed for preparing biological samples for nucleic acid amplification, the biological sample can readily be introduced into the tube without significant surface resistance, while then allowing the heat exchange characteristics of the volume of the biological sample to be altered by manipulation of the tube relative to a sample block of a thermocycler. That is, the mere insertion of the tube into such a sample block can cause the tube to deform elastically, so that the overall thickness of the biological sample that is heated becomes thinner, and more efficient for heat exchange (as compared with its original volume). Further, deformation of the tube facilitates improved contact between the tube and the sample block which improves heat transfer to a sample within the tube. Moreover, by virtue of a unique geometry, selection of materials and/or material processing, an improved sample tube is achieved that provides optical clarity for allowing improved light focus and transmission for excitation and detection of fluorophores as part of a real-time PCR analysis, without compromise to the heat exchange characteristics of the tube.

### DESCRIPTION OF THE DRAWINGS

FIG. 1 is a perspective view of an illustrative example of an illustrative tube of the present teachings.

FIG. 2 is a side profile view of the tube of FIG. 1.

FIG. 2A is a front view of the tube of FIG. 1.

FIG. 3 is a sectional view of a tip of the tube of FIG. 1 showing the major and minor axes.

FIG. 4A is a cross-sectional view of an illustrative example of a sample block showing the tube of FIG. 1 partially inserted into a sample block opening.

FIG. 4B is a cross-sectional view of the sample block of FIG. 4A showing the tube of FIG. 1 fully inserted into a sample block opening.

FIG. 4C is a cross-sectional view of the sample block of FIG. 4A showing the tube of FIG. 1 fully inserted into a sample block opening.

FIG. 5A is a perspective view of an illustrative example of a tip of a tube of the present teachings.

FIG. 5B is a side cutaway view along the minor axis of an illustrative example of a tip of a tube of the present teachings.

FIG. 5C is a front cutaway view along the major axis of an illustrative example of a tip of a tube of the present teachings.

FIG. 6 is a perspective view of another illustrative tip with regions denoted for opposing light transmission optics for a real-time PCR instrument.

FIG. 7 is front sectional view illustrating an example of a tip in an opposing relationship with optical fibers for transmitting light in a real-time PCR instrument.

FIG. 8 is a graph representation of a qPCR protocol using an exemplary tube in accordance with the present teachings.

### DETAILED DESCRIPTION

This application is related to and claims the benefit of the filing date of U.S. Provisional Application Ser. No. 61/947,697 filed Mar. 4, 2013, the contents of this application being hereby incorporated by reference for all purposes.

The explanations and illustrations presented herein are intended to acquaint others skilled in the art with the teachings, its principles, and its practical application. Those skilled in the art may adapt and apply the teachings in its numerous forms, as may be best suited to the requirements of a particular use. Accordingly, the specific embodiments of the present teachings as set forth are not intended as being exhaustive or limiting of the teachings. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description.

This application is also related to U.S. Provisional Application No. 61/681,879 filed Aug. 10, 2012 and U.S. Provisional Application. No. 61/752,494, filed Jan. 15, 2013. This application is also related to U.S. application Ser. Nos. 13/484,963 filed May 31, 2012 and Ser. No. 13/833,349 filed Mar. 15, 2013. The contents of the aforementioned applications are hereby incorporated by reference for all purposes.

The present teachings are predicated upon an improved sample tube for use in PCR sample amplification and real-time analysis. The present teachings pertain generally to an improved sample tube that exhibits relatively good heat exchange performance as well as optical transparency for light transmission of a sufficient level for excitation and detection of luminescing agents, fluorophores, or other light emitting agents as part of a real-time PCR analysis. The sample tube thus finds particularly attractive utility for polymerase chain reaction nucleic acid amplification protocols that employ repeated thermal cycling between hotter and cooler temperatures. The tube structure employs a relatively thin walled sample holding portion and a relatively thin walled substantially transparent sample tip.

In general, the tube of the present teachings employs a resiliently deformable structure that allows the tube to achieve intimate thermal communication (e.g., direct contacting communication) with a sample block that is the object of rapid heating and cooling. For instance, the sample block may be a silver-containing block that includes a plurality of elongated bores that have a generally oval transverse cross section along at least 50% their length. The tube also employs at least one portion of sufficient optical transparency and is molded into a specific shape so that luminescing agents, fluorophores, or other light emitting agents can be excited and detected therethrough as part of a real-time PCR analysis, such as an analysis made using an instrument in which excitation light, emission light or both are transmitted from a location beneath a tip of the tube (e.g., by an instrument in accordance with the teachings of co-pending U.S. application Ser. Nos. 13/833,349 (filed Mar. 15, 2013) and 61/840,755 (filed Jun. 28, 2013)).

Though larger volume tubes are also within the scope of the present teachings, the teachings herein envision a miniature tube for holding relatively small volumes of a biological sample (such as from about 10 μL to 100 μL; for example a sample volume of about 25 μL to 50 μL). As a result of such small volumes, the amount of luminescing agent, fluorophore, or other light emitting agent will be relatively small as well. By way of illustration, the concentration of the agent in the sample tube may be on the order of only about 10 to about 500 nanomolar (nM). It may be on the order of only about 50 to about 100 nM. With such a small sample volume and small concentration, the total amount of the luminescing agent, fluorophore, or other light emitting agent to be detected may range from 0.1 pmol to 50 pmol. It may be on the order of about 0.5 pmol to 10 pmol. Methods in accordance with the present teachings envision use of such agent in such concentrations. It will be recognized that the luminescing agent, fluorophore or other light emitting agent will typically be bound to an amplified target analyte (e.g., a nucleic acid or portion or fragment thereof). The action of binding to a target analyte may affect the amount of fluorescence of the luminescing agent, fluorophore or other light emitting agent. This difference in fluorescence may carry information regarding the quantity of the target analyte. Thus the amount of bound luminescing agent, fluorophore, or other light emitting agent may need to be detected in quantities lower than the total amount of luminescing agent, fluorophore, or other light emitting agent. The detection limit of bound luminescing agent, fluorophore, or other light emitting agent may be 10 times, 100 times, or even 1000 times lower than the total amount of luminescing agent, fluorophore, or other light emitting agent. By way of illustration, the detection limit may be as low as 0.01 pmol.

By virtue of the unique construction and method of manufacture of the sample tubes herein, the sample tubes are shaped to transmit sufficient light into and out of the tube so that the light emitting agent can be excited, and light from the resulting excited agent (albeit present in relatively low amounts), can be sufficiently detected by a real-time analysis instrument (e.g., by way of an optical fiber arrangement located generally opposite a substantially transparent portion of the tube). By virtue of the unique construction and method of manufacture of the sample tubes herein, it is possible to reliably and reproducibly detect (and be able to quantify an analyte) an excited light emitting agent through a substantially transparent portion of the sample tube that is smaller than about 7 mm², smaller than about 5 mm², or even smaller than about 3 mm². For example, the substantially transparent portion of the tube through which an excited light emitting agent may be reliably detected may range from about 0.3 to about 2 mm², about 0.5 to about 1.5 mm², or even about 0.7 to about 1 mm². The total area of the substantially transparent portion of the tube through which excitation light can be transmitted to excite one or a plurality of light emitting agents may be smaller than about 3 mm², smaller than about 1 mm², or even smaller than about 0.3 mm². For example, it may be in the range of about 0.05 to about 0.6 mm², about 0.1 to about 0.4 mm², or even about 0.15 to about 0.25 mm².

Turning now to a discussion of the construction of sample tubes of the present teachings, such teachings pertain generally to a polymeric sample tube having a body portion including a longitudinal axis and an outer wall generally circumscribing the longitudinal axis. The polymeric sample tube may include a closure portion, a strap integrally connected to the closure portion and being configured for defining a living hinge. The body portion may be integrally and hingedly connected with the closure portion by way of the strap. The body portion includes a head portion that has an opening through which a sample is dispensed, and a tapered sample portion having a first outer wall dimension. The head portion includes a positive stop portion. The positive stop portion may be located at an end of the head portion. The positive stop portion may be located prior to an end of the head portion. The positive stop portion may be wider than one or more portions adjacent the positive stop portion. The positive stop portion may be sufficiently wide so that it prevents the tube from entering into a sample block any further than desired. The body portion also includes at least one transparent portion that is adapted for transmitting light for excitation of a luminescing agent, a fluorophore or some other light emitting agent, and is also adapted for transmitting light emitted by a luminescing agent, a fluorophore or some other light emitting agent that has been excited and is coupled with an analyte of interest. The transparent portion may extend over all or only part of the sample portion.

As one example, the body portion may have a closed substantially transparent distal tip that is located at an end of the sample tube that is remote from the head portion. The body portion may include a wall structure may having an outer wall and an inner wall structure for defining a hollow cavity within which the sample resides as a sample volume after is dispensed through the head portion. The sample portion (which may be formed within or as part of the body portion) is generally elongated along the longitudinal axis and is configured for elastic deformation along at least a portion of its length, including in a direction that is generally transverse to the longitudinal axis. In this manner, it is envisioned that at least a portion of the wall structure compressively and resiliently deforms and engages a wall defining an opening in a sample block of a PCR amplification instrument, and a first outer wall dimension of the sample portion reduces to a smaller second outer wall dimension.

For improved focus of the light for excitation, the substantially transparent distal tip may be configured to include at least one concave dimple that projects generally inwardly within the interior of the sample portion and has a dimple depth relative to a tip end. It will be seen that the portion of the tube wall that defines the dimple will have a generally constant wall thickness. Thus, there will be both a projection of the tube wall into the sample portion, and a depression in the exterior of the tube tip. The dimple structure aids in focusing the light for excitation by minimizing spreading of the light. Thus, more excitation light is focused to the fluorophores leading to more light emitted from the fluorophores and detected by the detector.

The tube may be a molded structure (e.g., a structure made by injection molding) fabricated from a polymeric material including a thermoplastic that exhibits a melt flow rate of about 35 to about 60 g/10 min (per ASTM D-1238-10), a flexural modulus of about 900 to about 1400 MPa (per ASTM D-790A-10 (reported as 2% secant)), and a haze (per ASTM D-1003-11e1; for a section of about 1.1 mm thickness) below about 12%. The tube may be a molded structure fabricated from a material including a polyolefin that exhibits a melt flow rate of about 40 to about 55 g/10 min (per ASTM D-1238-10), a flexural modulus of about 1000 to about 1200 MPa (per ASTM D-790-10 (reported as 2% secant)), and a haze (per ASTM D-1003-11e1; for a section of about 1.1 mm thickness) below about 9%. By way of example, the tube may be a molded structure fabricated from a polymer consisting essentially of (e.g., it includes at least about 90 percent by weight of) a random polypropylene copolymer. The transparent portion of the tube will exhibit a haze (per ASTM D-1003-11e1) below about 12%, 9% or even below about 6%. Examples of illustrative commercially available polymeric materials useful herein include, without limitation, Total Petrochemicals Polypropylene 3847MR (Total Petrochemicals USA, Inc., Houston, Tex.); Braskam PP RP250 (M. Holland Company Northbrook, Ill.); Pro-fax RP448S (LyondellBasell Industries, Rotterdam, South Holland); Topas 5013S-04 (Topas Advanced Polymers GmhH, Frankfurt-Hochst, Germany); and FHR P9M7-056 (Hint Hills Resources, Wichita, Kans.).

Especially in the region of the tip (which may include or be defined within the substantially transparent portion) (e.g., from the tip end to about 2 mm from the tip end, but possibly also over at least about 50%, 70%, 90% or more of the length of the sample portion), the outer wall and the inner wall (34 and 36 respectively of FIG. 2) will define a wall thickness (t) that may be generally constant. For instance, it may have an average wall thickness and the maximum deviation from the average wall thickness will be less than about 30%, less than about 20% or even less than about 10%. By way of illustration, the sample tube may have an average wall thickness in the region of the tip (e.g., from the outside bottom of the tube to a distance of about 2 mm from the outside bottom of the tube) of about 0.05 to about 0.3 mm, or even about 0.1 to about 0.2 mm thick.

The sample tube may have a generally oval transverse sectional shape including a minor transverse axis with an inner width and an outer width and a major transverse axis with an inner length and an outer length. The phrase “generally oval” or “oval” as used herein, contemplates within its scope not only an oval geometry, but also an elliptical geometry, as well as an ovoidal geometry or another like rounded geometry having a major axis and an minor axis that differ in dimension.

Prior to any compressive and resilient deformation, the ratio of the inner width (w_{I}) of the minor axis of the tip to the inner length (l_{I}) of the major axis of the tip is about 1:5 to about 1:1.5. For example, the ratio of the inner width (w_{I}) of the minor axis of the tip to the inner length (l_{I}) of the major axis of the tip may be about 1:3. Prior to any compressive and resilient deformation, the ratio of the outer width (wₒ) of the minor axis of the tip to the outer length (lₒ) of the major axis of the tip may be about 1:5 to about 1:2. For example, prior to any compressive and resilient deformation, the ratio of the outer width (wₒ) of the minor axis of the tip to the outer length (lₒ) of the major axis of the tip may be about 1:2.3.

The sample tube may be tapered along the sample portion. For example, the sample portion may taper from an outer width (wₒ) of the minor axis at the positive stop portion to the tip in a ratio of about 2:1, or specifically about 2.3:1.4.

The sample tube may be characterized as having a generally slender sample portion. The ratio of the outer width (wₒ) of the minor axis of the tip to the length (lₛ) of the sample portion (stopping at the positive stop portion) may be about 1:15 to about 1:25 (e.g., it may be about 1:20). The ratio of the outer width (wₒ) of the minor axis of the tip to the length (lₛ) of the sample portion (including the entire head portion) may be about 1:15 to about 1:25 (e.g., it may be about 1:22.5).

As indicated, desirably, the sample tube of the present teachings will also include at least one dimple. The dimple will have a height relative to the tip end (i.e., the height is taking into account no inversion of the tube; conversely, it will have a dimple depth if the tube is inverted). It is envisioned that a ratio of the dimple height to the inner width (W_{I}) of the minor axis of the tip may be about 0.05:1 to about 0.3:1. More particularly, the ratio of the dimple height to the inner width (w_{I}) of the minor axis of the tip may be about 0.16:1. The ratio of the dimple height to the inner length (l_{I}) of the major axis of the tip may be about 0.05:3 to about 0.3:3. The ratio of the dimple height to the inner length (l_{I}) of the major axis of the tip is about 0.17:3.

Along the major axis, the upper edge of the head portion may have an outer width of about 6.5 mm and an inner width of about 5.7 mm. The lower edge of the head portion, adjacent the neck, may have an outer width of about 6.33 mm and an inner width of about 5.0 mm. The lower edge of the neck, adjacent the positive portion, may have an outer width of about 4.18 mm and an inner width of about 3.37 mm. The positive stop portion may have an outer width of about 4.06 mm. The top edge of the sample portion adjacent the positive stop portion may have an outer width of about 3.73 mm.

Along the minor axis, the upper edge of the head portion may have an outer width of about 5.09 mm and an inner width of about 4.32 mm. The lower edge of the head portion, adjacent the neck, may have an outer width of about 4.90 mm and an inner width of about 3.69 mm. The lower edge of the neck, adjacent the positive portion, may have an outer width of about 2.89 mm and an inner width of about 2.03 mm. The positive stop portion may have an outer width of about 2.7 mm. The top edge of the sample portion adjacent the positive stop portion may have an outer width of about 2.28 mm.

The distance from the tip to the positive stop portion may be between about 27 and 28 mm. The distance from the tip to the bottom edge of the neck may be between about 30 and 32 mm. The distance from the tip to the top of the tube (below the cap) may be between about 40 and 42 mm.

The present teachings also contemplate use of a tube as described. For example, the tubes herein may be employed to receive a quantity of a sample. The sample may be a biological specimen. Thus, it is possible that the tubes herein are employed to receive a sample for nucleic acid (e.g., DNA and/or RNA) amplification. The nucleic acid amplification may be performed in a thermocycler. For example, the tubes herein may be employed to amplify a sample for nucleic acid amplification in a thermocycler that has a sample block (optionally a solid metal sample block, such as a silver-containing sample block) that includes at least one bore defined by a wall having a generally oval transverse section along at least a portion of its length. An example of one suitable thermocycler is described in co-pending U.S. application Ser. No. 12/918,914. The sample block may have one or more openings for receiving light from one or more light sources via one or more optical fiber arrangements, and for transmitting light emitted by one or more light emitting agents contained within a sample tube or tubes in the sample block. The tubes may be employed in a step of inserting the tubes containing an analyte into a sample block having one or a plurality of bores therein so that contact with the walls causes the tubes to resiliently deform (such deformation may be temporary or permanent) so that heat exchange within the tube is more efficient than in the original configuration (e.g., prior to deformation during insertion into a bore) that received the sample. A step may be employed of transmitting light to the sample through the substantially transparent portion (e.g., the tip) to excite one or more light emitting agents associated with an amplified analyte (e.g., nucleic acid) of interest in the sample. Another step may be employed of detecting light emitted by the one or more light emitting agents. For example, one approach is to perform the real-time analysis using steps of transmitting light through the tip of the sample tube; that is, light for exciting a light emitting agent and/or light emitted by light emitting agent is transmitted through the tube tip and based solely upon the light transmitted through the tube tip, real-time analysis is performed.

The transmitting and detecting steps may employ discrete optical fiber arrangements adapted respectively for transmitting or detecting light. Such discrete optical fibers arrangements may be isolated relative to each other, and disposed generally opposite a predetermined portion of the sample tube. For example, an optical fiber arrangement may be arranged generally opposite a central region of the tube tip for detecting. There may be a step of disposing the dimple of such sample tube generally opposite the optical fiber arrangement adapted for detecting. Such a step may employ positioning the tube tip so that the optical fiber arrangement extends into the dimple (e.g., it crosses a plane of the tube tip). There may also be a step of positioning the tube so that transverse portions are generally opposite a plurality of optical fiber arrangements adapted for transmitting light to excite one or more light emitting agents in the sample tube.

There also is contemplated the use of the sample tubes herein in an instrument in which one or more optical fiber arrangements are employed for directing an excitation, light toward a sample, for receiving light emitted by the sample after excitation, or both. For instance, one preferred method contemplates use of an instrument in accordance with the teachings of co-pending U.S. application Ser. Nos. 13/833,349 (filed Mar. 15, 2013) and 61/840,755 (filed Jun. 28, 2013), both incorporated by reference for all purposes. In those applications, instruments are taught that employ an optical fiber arrangement for delivering an excitation light, and an optical fiber arrangement for receiving light emitted by an analyte coupled with an excited luminescing agent, fluorophore or other light emitting agent that has been excited. One or more of the optical fiber arrangements may be disposed beneath a sample that is held in a sample holder (e.g., a sample block including bores that are shaped so that they apply compressive forces to the wall structure defining the sample portion).

Accordingly, for use in the present teachings there is envisioned to be employed a tube tip (which may include or be formed within the substantially transparent portion) that is configured to oppose an optical fiber arrangement for providing a plurality of excitation light sources, to oppose an optical fiber arrangement for receiving light emitted from one or more excited light emitting agents contained within the sample portion, or both. The tube tip may thus be configured to oppose in a central region of the tip an optical fiber arrangement for receiving light emitted from one or more excited light emitting agents contained within the sample portion, and may be configured to oppose a plurality of optical fiber arrangements (e.g., two, three or more) for providing a plurality of excitation light sources on transversely opposing sides of the central region. In one particular approach, the tube tip may be configured to oppose a plurality of optical fiber arrangements for providing a plurality of excitation light sources including three optical fiber arrangements positioned generally in a triangular manner relative to each other.

Other features of the teachings herein are also possible. By way of illustration, the head portion may be dimensioned for frictionally engaging the closure portion. In this regard, the head portion may be dimensioned for frictionally engaging the closure portion and engaging the closure portion by way of a snap-fit or friction fit. The closure portion may be separately formed from the tube and/or separately attached to the tube. The head portion may be generally cylindrical. The head portion may be circular in shape or may be generally oval in shape. It may be generally tubular. It may have a substantially constant wall thickness along its length, about its circumference, or both. The head portion may have a generally circular transverse cross-section along its length that has an inner diameter of about 3 to about 4 mm. The head portion may have a generally oval transverse cross-section along its length that has an inner diameter of about 3 to about 4 mm. The head portion may have a generally circular outer diameter. The head portion may have a generally oval outer diameter. It may have an outer diameter of less than about 7 mm (e.g., about 5.5 to about 6.5 mm). The head portion may be formed for pipette loading. The head portion may be formed so that it has sufficient space to receive air pressure formed upon compression of the sample portion of the tube. The head portion may be located adjacent an intermediate portion (e.g., a juncture).

There may be an intermediate portion located between the head portion and sample portion. The diameter of the tube may increase in moving from the sample portion to the head portion such that the intermediate portion comprises the portion of the tube where the diameter expands rapidly. The intermediate portion may have a continuously variable slope around its circumference. The intermediate portion may have a consistent circumference along its length. The intermediate portion may define a neck having a tapered wall of one or more slopes as evidenced by multiple angles relative to the bottom of the intermediate portion where it intersects with the sample portion. The slopes may gradually and continually vary around the circumference of the neck portion. The intermediate portion may be integrally formed with the sample portion and head portion and may also include a smooth surface with no attachments or extensions.

Alternatively, the intermediate portion may be formed so that at least a portion of the tube is prevented from entering an opening in a sample block of a thermocycler. More specifically, the intermediate portion may define a neck having a diameter that exceeds the diameter of the sample portion so that the neck is prevented from entering an opening in a sample block. The intermediate portion may thus be formed to include a feature or attachment that acts as a stop to prevent the sample tube from entering into a sample block further than desired.

The sample portion may have a length that is longer than that of the head portion. For example, the sample portion may have a length that is greater than the length of the head portion by a factor of at least about 3. The length (lₛ) of the sample portion may be at least about 20 mm. For example, it may be about 22 to about 35 mm, about 25 to about 30 mm or about 27 mm. The sample portion may have a maximum outer width (wₒ) in an open, non-compressed state, of below about 5 mm, or even below about 4 mm. For example, it may have an maximum outer width of about 3.7 mm. Overall tube lengths may be about 30 to about 50 mm (e.g., about 40 mm).

The tube may have about a 0.1 to about 0.4 (e.g., about 0.2 mm) radius in the external tube tip wall when transitioning from the vertical tube body walls to the bottom of the tube tip. It may have about a 0.02 to about 0.07 (e.g., about a 0.05 mm) radius on the internal transition from the vertical tube body walls to the bottom of the tube tip.

The sample portion, along substantially the entirety of its length, may have a transverse cross-section outer profile that includes a transverse minor axis and a transverse major axis. The sample portion may have an outer profile that tapers along the longitudinal axis so that it narrows as it approaches the closed end of the tube (e.g., the end opposing the head portion). For example, the sample portion may have an outer profile that tapers generally continually along substantially the entirety of the length of the sample portion so that it narrows in at least one axis transverse to the longitudinal axis from a first outer wall dimension to a second outer wall dimension that is less than about one two thirds (e.g., about one half) of the first outer wall dimension as it approaches the closed end of the tube. The outer profile may taper more rapidly in at least one section to create at least one neck feature on the outer profile to aid in positioning the tube in the same depth within each bore of the sample block.

The sample portion may be defined by an interior wall that has a generally oval cross section in a direction transverse to the longitudinal axis, for substantially the entirety of the length of the closed-ended hollow sample portion. By way of example, the sample portion may be defined by an interior wall that has a generally oval cross section that includes a minor axis and a major axis that is generally perpendicular to the minor axis, with each axes being oriented in a direction transverse to the longitudinal axis and having a dimension, for substantially the entirety of the length of the closed-ended hollow sample portion. The interior wall of the sample portion may have a taper along the longitudinal axis for the major axis which is less than 2° (e.g., about 0.98°) to assist in the core pin removal and to allow long pipette tips to reach the bottom of the sample portion without having too much sample volume capacity loss by using a large taper angle (e.g. above about 2°). The interior wall of the sample portion may have a taper along the longitudinal axis for the minor axis which is less than 2° (e.g., about 1.83°) to assist in the core pin removal and to allow long pipette tips to reach the bottom of the sample portion.

As can be appreciated, the sample tube portion may thus be configured so that during the compressive engagement within the sample block, an interior volume per unit length of the sample tube portion at the region proximate the distal end does not exceed an interior volume per unit length of the sample tube located more proximate to the head portion. The sample tube may be configured so that, during the compressive engagement, any deflection of the sample portion occurs relative to a generally fixed pivot region. The sample tube may be configured so that, during the compressive engagement, any deflection of the sample portion occurs relative to a generally fixed pivot region and the amount of angular deflection is less than about 45° relative to the longitudinal axis. The sample tube may be configured so that, during the compressive engagement, any deflection of the sample portion occurs relative to a generally fixed pivot region and the amount of angular deflection is less than about 90° relative to the longitudinal axis. The sample tube may be configured so that, during the compressive engagement, any deflection of the sample portion occurs relative to a generally fixed pivot region and the amount of angular deflection is less than about 15° relative to the longitudinal axis. The sample tube may be configured so that, during the compressive engagement, direct contact between opposing inner wall portions of the sample portion is avoided. Alternatively, during the compressive engagement, direct contact between opposing inner wall portions of the sample portion may occur and may promote sufficient heating and cooling cycles of a sample. The sample tube may be configured so that, during the compressive engagement, the closure remains in a closed and substantially sealed relationship with the head portion.

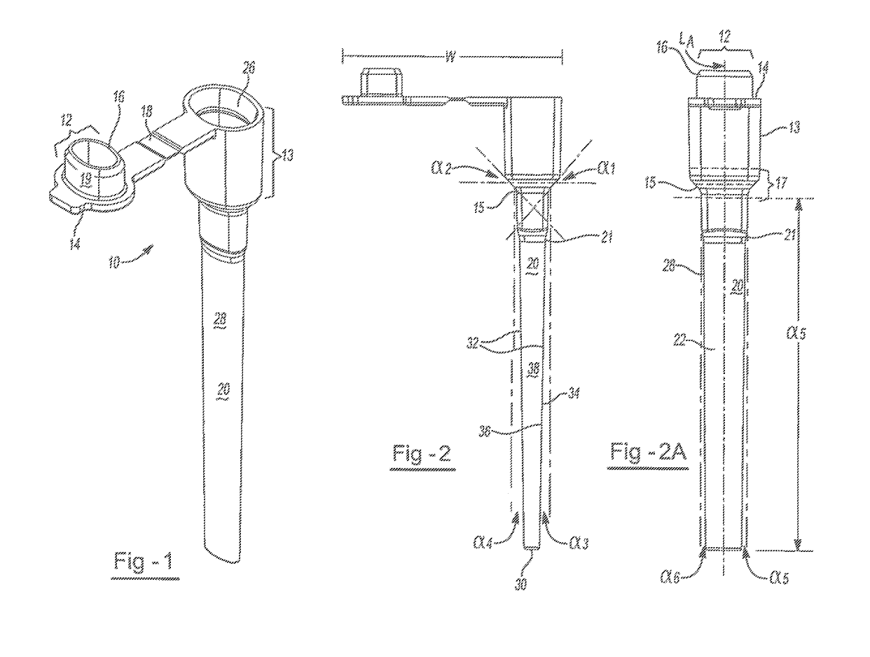

Turning now to the drawings to illustrate examples of embodiments of the present teachings. As shown for example in FIGS. 1, 2 and 2A, a sample tube 10 is shown having a closure portion 12 (which itself may include a tab portion 14, and an adjoining plug portion 16). A strap 18 integrally connects to the closure portion 12 and is configured for defining a living hinge. The tube includes a head portion 13 to which the closure portion 12 is attached via the strap 18. In the open position (e.g., when the closure is not located within the head portion), the closure portion and head portion may combine to form an open tube width (W) (see FIG. 2) that includes the combined width of the closure portion 12, strap 18, and head portion 13. The closure portion 12 may have a side wall 19 that matingly engages an inner wall of the head portion 13. The side wall 19 may have a length from the tab portion to a distal edge of about 1.5 to about 4 mm (e.g., about 2.5 mm). The side wall 19 may be slightly angled (such as from about 1 to about 5), e.g., about −2°), over some or all of its length, relative to the longitudinal axis.

An intermediate portion 17 may be located in between the head portion 13 and body portion 20. The intermediate portion 17 may define a neck 15 having a tapered wall of one or more slopes as evidenced by angles (e.g., α1, α2) relative to the bottom of the intermediate portion 17 where it intersects with a sample portion 28. The slopes may gradually and continually vary around the circumference of the neck portion. The neck may be located adjacent a positive stop portion 21. The positive stop portion includes a width that is wider than that of any diameter of the sample portion so that the tube is prevented from travelling deeper into a sample block bore than desired. The largest width of the positive stop portion may still be smaller than the largest width of any of the neck.

The body portion 20 has a longitudinal axis (L_{A}) (as shown at FIGS. 2A and 4C) and an outer wall 22 generally circumscribing the longitudinal axis. The body portion includes the head portion 13 that has an opening 26 through which a sample is dispensed and/or received, and a sample portion 28 having a first outer wall dimension (OWD1) (as shown at FIG. 4A). The sample portion includes a closed distal end 30 (which may include a dimple), and a wall structure 32 that includes an outer wall 34 and an inner wall 36 that defines a hollow cavity 38, within which the sample resides as a sample volume after is dispensed through the head portion. As seen, the closed-ended hollow sample portion is generally elongated along the longitudinal axis. Over at least a portion of the length of the sample portion, the outer wall 34 is tapered. It is tapered at an angle α3 and α4 as shown in FIG. 2. It may also be tapered at an angle α5 or α6 as shown in FIG. 2A. The angles α3 and α4 may be generally about the same, and may range from about 0.01 to about 20° (e.g., about 0.4 to about 5°). The angles α5 and α6 may be generally about the same, and may range from about 0.01 to about 10° (e.g., about 0.2 to about 4°; for instance it may be about 0.5°).

With reference to FIGS. 4A-4C, it is also seen how at least the sample portion is configured for elastic deformation along a portion of its length. FIG. 4A shows the tube prior to deformation by insertion into a sample block 24, while FIG. 48 shows the tube upon deformation when inserted into the sample block 24. Specifically, FIG. 4C illustrates how, when a force is applied to the tube from a direction that is generally transverse to the longitudinal axis (such as a force realized when inserting such tube into an opening of a sample block 24), at least a portion of the wall structure 32 compressively and resiliently deforms and engages a wall 25 defining the opening in the sample block. The first outer wall dimension of the sample portion reduces to a smaller second outer wall dimension (OWD2). During compression, a first internal diameter (D₁) across the tube may increase, while a second internal diameter (D₂) that lies perpendicular to the first diameter may decrease.

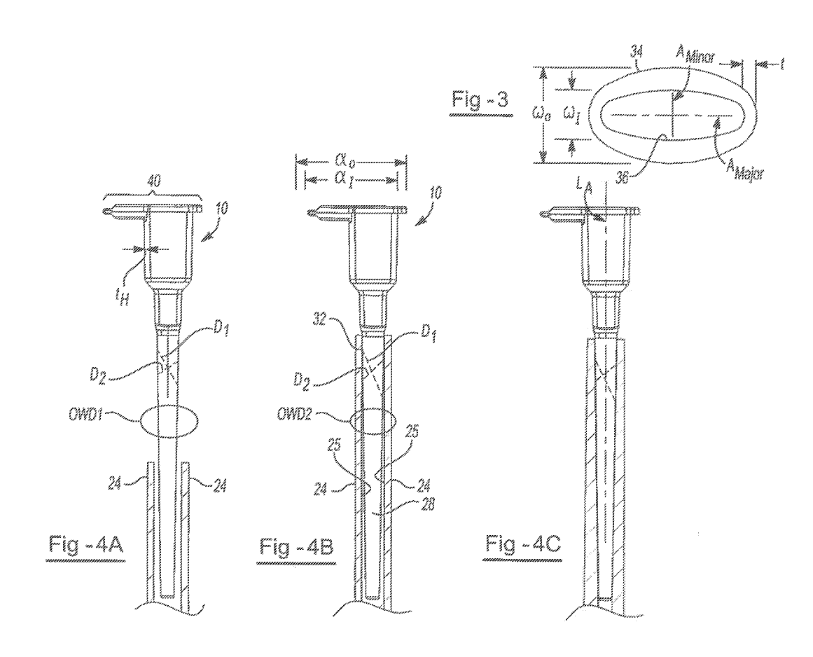

As seen, the head portion frictionally engages the closure by way of a snap-fit connection structure 40. The head portion may have a substantially constant wall thickness (t_{H}) along its length, about its circumference, or both. As shown for example in FIG. 3, the body portion as well as any tip portion may have a generally oval transverse cross-section along its length that has a major axis (Aₘₐⱼₒᵣ) and a minor axis (Aₘᵢₙₒᵣ). The tube may have an inner length (l_{I}) and an outer length (Iₒ). The tube may have an inner width (w_{I}) in the direction of the minor axis and an outer width (wₒ). Especially in the region of the tip (e.g., from the tip end to about 3 mm from the tip end, but possibly also over at least about 50%, 70%, 90% or more of the length of the sample portion), the outer wall 34 and the inner wall 36 will define a wall thickness (t) that may be generally constant. For instance, it may have an average wall thickness and the maximum deviation from the average wall thickness will be less than about 30%, less than about 20% or even less than about 10%.

Prior to any compressive and resilient deformation, the ratio of the inner width (w_{I}) of the minor axis of the tip to the inner length (I_{I}) of the major axis of the tip is about 1:5 to about 1:1.5 (e.g., about 1:2.8). Prior to any compressive and resilient deformation, the ratio of the outer width (wₒ) of the minor axis of the tip to the outer length (lₒ) of the major axis of the tip may be about 1:5 to about 1:2 (e.g., about 1:2.3).

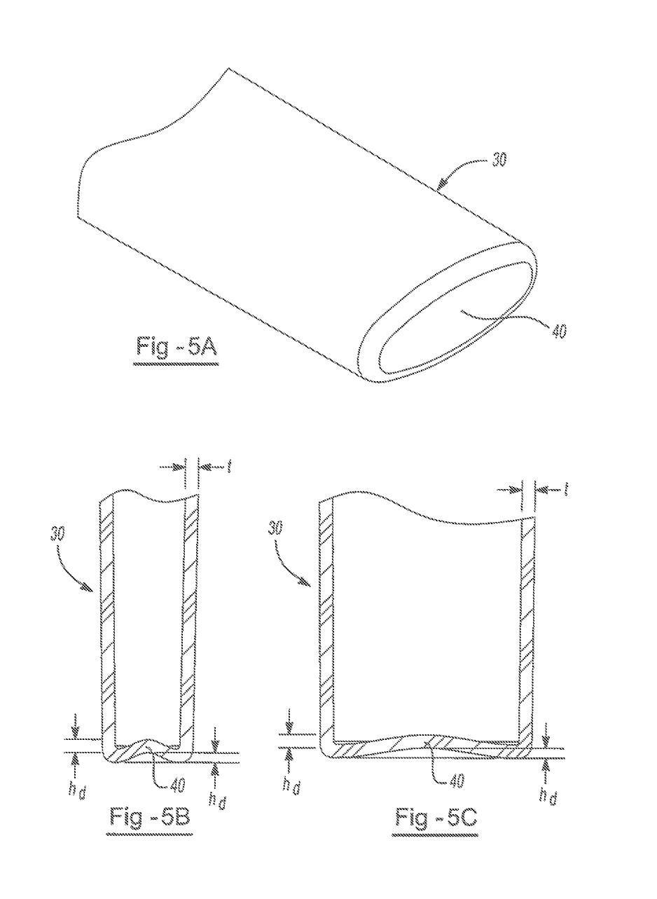

As shown in more detail in FIGS. 5A-5C, the tube tip 30 may include a dimple 40. The dimple projects inwardly toward the head portion of the tube. The dimple has a height (h_{d}). The dimple height may be about 0.01 mm to about 0.5 mm (e.g. about 0.15 mm). It can alternatively be stated that the dimple will have a depth relative to the tip end (i.e., the depth is taking into account an inversion of the tube). It is envisioned that a ratio of the dimple height to the inner width (w_{I}) of the minor axis of the tip may be about 0.05:1 to about 0.3:1 (e.g., about 0.15:1). The ratio of the dimple height to the inner length (l_{I}) of the major axis of the tip may be about 0.05:3 to about 0.3:3 (e.g., about 0.15:3). It is seen that the dimple of this example, and more generally other tubes in accordance with the teachings may be arcuate over its entire portion.

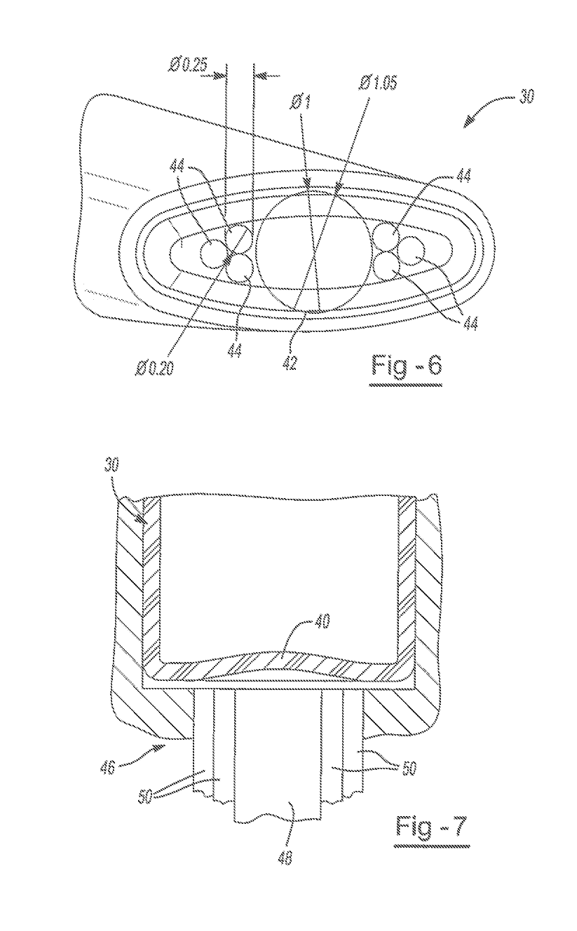

Referring to FIG. 6, there is depicted an alternative structure in which there is a dimple that includes a generally flat portion. The dimple is configured to include a central portion 42 of sufficient size (such as about 0.05 to about 1.5 mm diameter, e.g., about 1 mm diameter) that it can oppose an optical fiber arrangement or other light collection means adapted to receive light emitted by a luminescing agent, a fluorophore, or other light emitting agent contained in the sample portion of the tube. It also includes a plurality of triangularly arranged portions of sufficient size (such as about 0.05 to about 0.4 mm diameter, e.g., about 0.2 mm) transversely flanking the central portion. These latter portions are adapted to oppose one or more light sources for exciting luminescing agent, a fluorophore, or other light emitting agent contained in the sample portion of the tube. The teachings of this alternative embodiment also find similar application as the embodiment of FIGS. 5A-5C, as will be seen in FIG. 7.

Referring to FIG. 7, it is seen how the central portion and flanking portions generally oppose an emission optical fiber arrangement 48 and a plurality of excitation optical fiber arrangements 50, which may be isolated relative to each other, such as by use of a sheath. As mentioned, the embodiments of either FIGS. 5A-5C or FIG. 6 can be used in an arrangement as shown in FIG. 7.

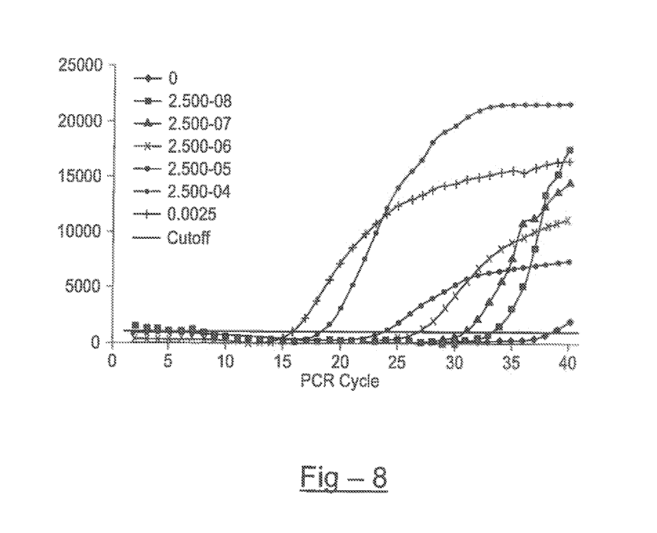

FIG. 8 depicts a graph showing the result of a qPCR protocol using the tube described herein Specifically, the protocol utilized an EBC gene primer set with a TAMRA probe at different dilutions. The qPCR program had a run time of 20 min using the tubes in accordance with the present teachings.

The dimensions shown in the drawings are incorporated by reference herein as illustrative examples of the teachings. The relative proportions shown in the drawings are likewise incorporated by reference herein even if not expressly recited in this description. However, the teachings are not limited solely to the embodiments and dimensions shown in the drawings.

The head portion is preferably integrally formed with the sample portion so that both the head portion and sample portion have a smooth surface with the only attachment or projection extending from either the head portion or sample portion being the closure portion. The head portion and sample portion may be integrally formed, but may be formed with a feature located intermediate the head portion and sample portion that acts as a stop to assist in locating the tube in a desired location within an opening during use. The diameter of the tube may expand in moving from the sample portion to the head portion to form the intermediate portion. The sample portion, the head portion, the closure portion or any combination thereof may be formed of a single layer of polymeric material. The tube may be substantially free of a triangular shaped closed end. The interior of the sample portion may form a smooth surface containing no additional elements (e.g., openings, receptacles, vessels, extensions, attachments, ridges) within the sample portion. The exterior of the sample portion may form a smooth surface containing no additional elements (e.g., openings, receptacles, vessels, extensions, attachments, ridges) within the sample portion. The sample portion may also be substantially free of any openings (e.g., ports). The sample portion may include only flexible walls and may be free of any rigid walls or rigid wall portions. The sample portion may include only rigid walls and may be free of any flexible walls or flexible wall portions. The sample tube tip may be free of any thickened section. It may be free of any convex surface within its central region.

When the closure portion is located into the sample portion to seal the tube, the top of the closure portion may be substantially flat with no attachments or extensions located on the closure portion. The closure portion may include a membrane located thereon to allow for access into the tube. Alternatively, the closure portion may be substantially free of any membrane. The closure portion may have an open position and a closed position. The closure portion may also be substantially free of any moving parts. More specifically, the closure portion may be substantially free of any parts to assist the closure portion in securely closing the tube. The strap connecting the closure portion to the head portion is preferably flexible with no means for securing the head portion in an open position or partially open position. The strap portion may also be free of substantial rigidity such that the strap will be unable to support the tube if any attempt is made to rest the tube on the strap or closure portion. More specifically, the tube may be free of any mechanism by which the tube can be supported in an upright position without the assistance of a separate holder. The head portion may include a textured surface. The textured surface may be adapted to receive printed or written information to identify patient information for a sample received within the tube.

The tube may be a fixed oval shape which may not be deformable. The sample portion may be substantially free of defined edges. The sample portion may receive non-biological samples. The sample portion, closure portion, positive stop portion, and/or head portion may receive identifying information, which may include an RFID code, NFC code, barcode, 2D barcode, OR code, clickable paper, or other unique computer recognizable image. The head portion may be substantially rigid so that it does not deform.

Multiple tubes may be connected together in a tube bundle. There may be 2, 4, 6, 8, or even 10 or more tubes connected in a single bundle. The tube bundle may have a spacing between tubes of about 3 mm to about 10 mm (e.g., about 7.05 mm). There may be a larger spacing between some tubes of about 5 mm to about 12 mm (e.g., about 8 mm) to separate the tubes into groups of 4 tubes. The individual tubes in the tube bundle may each have a unique RFID code. NFC code, barcode, 20 barcode, OR code, clickable paper, or other unique computer recognizable image. The tube bundle and/or groups of 4 individual tubes in a tube bundle may have a unique RFID code, NFC code, barcode, 2D barcode, OR code, clickable paper, or other unique computer recognizable image.

The tube bundle may be a single moldable part consisting of tubes connected by thermoplastic between the head of each tube. The tube bundle may be a single moldable part consisting of tubes connected by a thermoplastic between the closure portion of each tube. The tube bundle may consist of tubes connected together by placing individual tubes in a separate tube carrier which may be a moldable thermoplastic or similar material. The tube carrier may have 2, 4, 6, 8, or even 10 or more slots in which to hold individual tubes. The tube bundle may consist of individual tubes which snap-fit through their head portion of the tube into a strip of thermoplastic with multiple plugs such as 2, 4, 6, 8 or even 10 or more plugs which matingly engage an inner wall of the head portion of each individual tube. The tube bundle created with a strip of multiple plugs may matingly snap-fit into individual tubes each with their own hingedly connected lid, or may matingly snap-fit into individual tubes which have been molded without their hingedly connected lid.

As to all of the foregoing general teachings, as used herein, unless otherwise stated, the teachings envision that any member of a genus (list) may be excluded from the genus; and/or any member of a Markush grouping may be excluded from the grouping.

Unless otherwise stated, any numerical values recited herein include all values from the lower value to the upper value in increments of one unit provided that there is a separation of at least 2 units between any lower value and any higher value. As an example, if it is stated that the amount of a component, a property, or a value of a process variable such as, for example, temperature, pressure, time and the like is, for example, from 1 to 90, preferably from 20 to 80, more preferably from 30 to 70, it is intended that intermediate range values such as (for example, 15 to 85, 22 to 68, 43 to 51, 30 to 32 etc.) are within the teachings of this specification. Likewise, individual intermediate values are also within the present teachings. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner. As can be seen, the teaching of amounts expressed as “parts by weight” herein also contemplates the same ranges expressed in terms of percent by weight. Thus, an expression in the Detailed Description of the invention of a range in terms of at “‘x’ parts by weight of the resulting polymeric blend composition” also contemplates a teaching of ranges of same recited amount of “x” in percent by weight of the resulting polymeric blend composition.”

Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints. Concentrations of ingredients identified in Tables herein may vary ±10%, or even 20% or more and remain within the teachings.

The disclosures of all articles and references, including patent applications and publications, are incorporated by reference for all purposes. The term “consisting essentially of” to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of, or even consist of the elements, ingredients, components or steps. Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps.

It is understood that the above description is intended to be illustrative and not restrictive. Many embodiments as well as many applications besides the examples provided will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. The omission in the following claims of any aspect of subject matter that is disclosed herein is not a disclaimer of such subject matter, nor should it be regarded that the inventors did not consider such subject matter to be part of the disclosed inventive subject matter.

What is claimed is:

1. A sample tube comprising:
a body portion having a longitudinal axis and an outer wall generally circumscribing the longitudinal axis, the body portion including a tapered sample portion having a first outer wall dimension and including a closed substantially transparent distal tip, the sample portion being generally elongated along the longitudinal axis and being configured for elastic deformation along at least a portion of its length;
wherein the substantially transparent distal tip is configured to include a concave dimple that projects generally inwardly within the interior of the sample portion and has a dimple height relative to a tip end.

2. The sample tube of claim 1, wherein the tube is a molded structure fabricated from a polymer consisting essentially of a random polypropylene copolymer.

3. The sample tube of claim 1, wherein the tube is a molded structure fabricated from a polymeric material including a thermoplastic that exhibits a melt flow rate of about 35 to about 60 g/10 min (per ASTM D-1238-10), a flexural modulus of about 900 to about 1400 MPa (per ASTM D-790A-10 (reported as 2% secant)), and a haze (per ASTM D-1003-11e1; for a section of about 1.1 mm thickness) below about 12%.

4. The sample tube of claim 1, wherein the substantially transparent distal tip has an average wall thickness of about 0.05 to about 0.3 mm.

5. The sample tube of claim 1, wherein the substantially transparent distal tip has a generally oval transverse sectional shape including a minor transverse axis with an inner width and an outer width and a major transverse axis with an inner length and an outer length.

6. The sample tube of claim 5, wherein, prior to any compressive and resilient deformation, the ratio of the inner width of the minor axis of the tip to the inner length of the major axis of the tip is about 1:5 to about 1:1.5.

7. The sample tube of claim 5, wherein, prior to any compressive and resilient deformation, the ratio of the outer width of the minor axis of the tip to the outer length of the major axis of the tip is about 1:5 to about 1:2.

8. The sample tube of claim 5, wherein the sample portion along the minor axis tapers from a maximum transverse outer dimension to the outer width of the tip in a ratio of about 3:1 to about 2:1.

9. The sample tube of claim 5, wherein the ratio of the dimple height to the inner width of the minor axis of the tip is about 0.05:1 to about 0.3:1.

10. The sample tube of claim 5, wherein the ratio of the dimple height to the inner length of the major axis of the tip is about 0.05:2.8 to about 0.3:2.8.

11. The sample tube of claim 1, wherein the distal tip is configured to oppose an optical fiber arrangement for providing a plurality of excitation light sources.

12. The sample tube of claim 1, wherein the distal tip is configured to oppose an optical fiber arrangement for receiving light emitted from one or more excited fluorophores contained within the sample portion.

13. The sample tube of claim 1, wherein the distal tip is configured to oppose in a central region of the tip an optical fiber arrangement for receiving light emitted from one or more excited luminescing agents, fluorophores or other light emitting agents contained within the sample portion, and is configured to oppose a plurality of optical fiber arrangements for providing a plurality of excitation light sources on transversely opposing sides of the central region.

14. The sample tube of claim 1, wherein the distal tip is configured to oppose a plurality of optical fiber arrangements for providing a plurality of excitation light sources including three optical fiber arrangements, or multiple groupings of three optical fiber arrangements positioned generally in a triangular manner relative to each other.

15. The sample tube of claim 1, wherein the tube is sufficiently flexible in a direction that is generally transverse to the longitudinal axis so that at least a portion of the wall structure compressively and resiliently deforms and engages a wall defining an opening in a sample block of a polymerase chain reaction amplification device, and the first outer wall dimension of the sample portion reduces to a smaller second outer wall dimension.

16. The sample tube of claim 1, wherein the concave dimple focuses light to one or more fluorophores such that at least 1.5 times the amount of light enters the detection fibers as compared to the light that would enter the detection fibers without the concave dimple.

17. The sample tube of claim 1, wherein the concave dimple focuses light to one or more fluorophores such that at least 5 times the amount of light contacts the fluorophores as compared to the light that would contact the fluorophores without the concave dimple.

18. The sample tube of claim 1, wherein the concave dimple height is from 0.01 mm to 0.5 mm.

19. The sample tube of claim 1, wherein the concave dimple height is from 0.1 mm to 0.2 mm.

20. A sample tube comprising:
a body portion having a longitudinal axis and an outer wall generally circumscribing the longitudinal axis, the body portion including a tapered sample portion having a first outer wall dimension and including a closed substantially transparent distal tip, the sample portion being generally elongated along the longitudinal axis and being configured for elastic deformation along at least a portion of its length;
wherein the substantially transparent distal tip is configured to include a concave dimple that projects generally inwardly within the interior of the sample portion and has a dimple height of from 0.1 mm to 0.2 mm relative to a tip end; and
wherein the distal tip is configured to oppose an optical fiber arrangement for receiving light emitted from one or more excited fluorophores contained within the sample portion.
